(19) United States
(12) Patent Application Publication
(10) Pub. No.: US 2026/0117320 A1
(43) Pub. Date: Apr. 30, 2026

(54) 40K GENOME-WIDE LIQUID CHIP FOR SPRUCE AND USE THEREOF

(71) Applicant: Research Institute of Forestry, CAF, Beijing (CN)

(72) Inventors: Junhui WANG, Beijing (CN); Miaomiao ZHANG, Beijing (CN); Xiaowei YANG, Beijing (CN); Guijuan YANG, Beijing (CN); Sanping AN, Beijing (CN)

(21) Appl. No.: 18/814,960

(22) Filed: Aug. 26, 2024

(30) Foreign Application Priority Data
Dec. 12, 2023 (CN) 202311705879.5

(51) Int. Cl. C12Q 1/6895

(52) U.S. Cl. CPC C12Q 1/6895; C12Q 2600/13

(57) ABSTRACT

The present invention belongs to the field of genetic and molecular breeding technology, specifically relates to a 40K genome-wide liquid chip for Picea abies L. Karst (Norway spruce) and use thereof. The present invention provides a molecular marker combination for genotyping of Picea abies L. Karst, and the molecular marker combination comprises 40,000 SNPs. Based on the above 40,000 SNPs, the present invention further provides a 40K genome-wide liquid chip for Picea abies L. Karst, the 40K genome-wide liquid chip for Picea abies L. Karst provides uniform genome coverage, abundant functional SNPs, low cost, and simple bioinformatical analysis. This genomic liquid chip may be used in genotyping of spruce, conservation of germplasm resources, and study of molecular breeding, which facilitates to advance molecular marker-assisted breeding and genomic selection breeding, shortens the breeding cycle of forest trees, and improves the selection accuracy and breeding efficiency.

### CROSS-REFERENCE TO RELATED APPLICATION

This patent application claims the benefit and priority of Chinese Patent Application No. 202311705879.5 filed with the China National Intellectual Property Administration on Dec. 12, 2023, the disclosure of which is incorporated by reference herein in its entirety as part of the present application.

### TECHNICAL FIELD

The present disclosure belongs to the field of genetic and molecular breeding technology, specifically relates to a 40K genome-wide liquid chip for Picea abies (Picea abies 40K genome-wide liquid chip) and use thereof.

### BACKGROUND

Spruce (Picea asperata Mast) is a tree species with the highest stock volume in the world, and it is the main forest tree species in the temperate and cool temperate alpine areas of China. Picea abies L. Karst (Norway spruce) has the nature of excellent material, good trunk shape, and fast growth and shows strong adaptability and great production potential in China.

The emergence of molecular marker-assisted breeding and genomic selection technology can accelerate the process of forest tree breeding and improve the selection accuracy and efficiency, which are cutting-edge means for molecular breeding of spruce. However, spruce has a huge genome size of up to 20-30 Gb, which contains a large number of repeats and polymorphisms, and lacks high-quality reference genomes, posing a great challenge to the development of reliable single nucleotide polymorphisms (SNPs) that uniformly cover the genome. Although there are 50K solid-phase chips developed for Picea abies L. Karst in this field at present, the solid-phase chips involve a combination of fixed sites, and it is not possible to add or delete sites when used, which has limitations in use. Therefore, there is an urgent need to establish an efficient, low-cost, and standardized genotyping technology to break through the bottleneck that restricts the molecular breeding of spruce.

### SUMMARY

An object of the present disclosure is to provide a 40K genome-wide liquid chip for Picea abies L. Karst and use thereof. The 40K genome-wide liquid chip for Picea abies L. Karst can be used in the genotyping of Picea abies L. Karst, genetic breeding, and conservation of germplasm resources and has high detection efficiency, low cost, and wide application prospects.

The present disclosure provides a molecular marker combination for genotyping of Picea abies L. Karst, where the molecular marker combination includes 40,000 SNPs, and information on the 40,000 SNPs is shown in Table 1.

The present disclosure also provides a molecular marker segment including the molecular marker combination described above. Each of the molecular marker segments includes 40,000 marker segments, and each of the marker segments includes a nucleotide sequence of 100 bp upstream and a nucleotide sequence of 100 bp downstream an SNP, where the SNP is one of the 40,000 SNPs included in the molecular marker combination described in the above technical solution.

In one embodiment of the present disclosure, the molecular marker segment includes 74,687 multiple single nucleotide polymorphisms (mSNPs).

The present disclosure also provides a 40K genome-wide liquid chip for Picea abies L. Karst, where the 40K genome-wide liquid chip for Picea abies L. Karst includes the molecular marker combination in the technical solution described above.

The present disclosure also provides the use of the molecular marker combination, the marker segment, or the 40K genome-wide liquid chip for Picea abies L. Karst described in the above technical solutions in genotyping of Spruce.

The present disclosure also provides the use of the molecular marker combination, the marker segment, or the 40K genome-wide liquid chip for Picea abies L. Karst described in the above technical solutions in the conservation of germplasm resources.

The present disclosure also provides the use of the molecular marker combination, the marker segment, or the 40K genome-wide liquid chip for Picea abies L. Karst described in the above technical solutions in genetic breeding of spruce.

In one embodiment of the present disclosure, the spruce genetic breeding includes one or more selected from the group consisting of genetic evaluation of germplasm resources, genetic structure analysis of populations, identification of kinship, genetic mapping of quantitative traits, genome-wide association study, and genome-wide selection study.

### BENEFICIAL EFFECTS

The present disclosure provides a molecular marker combination for genotyping of Picea abies L. Karst. The molecular marker combination includes 40,000 SNPs, and the 40K genome-wide liquid chip for Picea abies L. Karst includes 40,000 SNPs, including 170 background SNPs, of which 120 have biological functions, and the rest are transcription factors. Among them, there are 44 SNPs associated with germination, 44 markers associated with wood properties and anatomical traits, 10 markers associated with growth traits such as tree height and diameter at breast height, and 22 insect resistance markers, covering about 45 traits commonly seen in Picea abies L. Karst.

Based on the above 40,000 SNPs, the present disclosure also provides a 40K genome-wide liquid chip for Picea abies L. Karst that evenly covers the genome. The 40K genomic liquid chip has the advantages of rich functional SNPs, low cost, and simple bioinformatical analysis. It may be used in genotyping and conservation of germplasm resources for a variety of spruce including Picea abies L. Karst, as well as in molecular breeding studies such as genetic evaluation of germplasm resources, genetic structure analysis of populations, identification of kinship, genetic mapping of quantitative traits (QTL), genome-wide association study (GWAS), and genomic selection (GS). It helps to advance molecular marker-assisted breeding and genomic selection breeding, shortens the forest tree breeding cycle, improves the selection accuracy and breeding efficiency, fills the gap in domestic liquid chips for forest tree breeding, and serves as the basis for clarifying the spruce genetic resources and accelerating the process of spruce genetic improvement to realize efficient breeding and promotion the innovation ability in spruce seed industry.

### BRIEF DESCRIPTION OF THE DRAWINGS

In order to illustrate embodiments of the present disclosure or the technical solution in the prior art more clearly, the accompanying drawings that need to be used in the embodiment will be briefly described below.

FIG. 1 is a map of the chromosomal distribution of the core SNPs in the 40K genome-wide liquid chip for Picea abies L. Karst in Example 1.

FIG. 2 is a map of the minor allele frequency (MAF) distribution of the core SNPs in the 40K genome-wide liquid chip for Picea abies L. Karst in Example 1.

FIG. 3 shows the annotation information of the core SNPs in the 40K genome-wide liquid chip for Picea abies L. Karst in Example 1.

FIG. 4 shows the chromosomal distribution of mSNP marker segments in the 40K genome-wide liquid chip for Picea abies L. Karst in Example 2.

FIG. 5 shows the principal component analysis for the Picea abies L. Karst germplasm resource population in Application Example 1.

FIG. 6 shows the phylogenetic tree analysis for the Picea abies L. Karst germplasm resource population in Application Example 1.

FIG. 7 shows the population structure analysis for the Picea abies L. Karst germplasm resource population in Application Example 1.

FIG. 8 shows GWAS analysis of the leaf bud grade of the Picea abies L. Karst under different models in Application Example 2.

FIG. 9 shows the quantile-quantile (QQ) plot under the composite multivariate linear model (CMLM) model in Application Example 2.

FIG. 10 shows the QQ plot under the fixed and random model circulating probability unification (FarmCPU) model in Application Example 2.

FIG. 11 shows the QQ plot under the Bayesian-information and linkage-disequilibrium iteratively nested keyway (BLINK) model in Application Example 2.

FIG. 12 shows the QQ plot under the mixed linear model (MLM) in Application Example 2.

### DETAILED DESCRIPTION OF THE EMBODIMENTS

The present disclosure provides a molecular marker combination for genotyping Picea abies L. Karst, where the molecular marker combination includes 40,000 SNPs, and information about the 40,000 SNPs is shown in Table 1:

Lengthy table referenced here
US20260117320A1-20260430-T00001
Please refer to the end of the specification for access instructions.

The reference genome used is the genome indicated in Nystedt B, Street N R, Wetterbom A, et al. The Norway spruce genome sequence and conifer genome evolution [J]. Nature, 2013, 497(7451): 579-584. In the present disclosure, the molecular marker combination retains 24,192 SNPs from the solid-phase chip for the foreign Picea abies L. Karst 50K, and 26,076 SNPs are optimized. The optimized SNPs account for 51.87% of the original SNPs. At the same time, 218 new QTL SNPs are significantly associated with traits ranging from wood property of Picea abies L. Karst, pest resistance, tree height, and diameter at breast height. In the end, a total of 40,000 high-quality SNPs are obtained. In the present disclosure, the 40,000 SNPs are evenly distributed across the Picea abies L. Karst genome, and the minor allele frequency ranged between 0.0 and 0.5, mainly between 0.05 and 0.2. Moreover, 72.44% of the SNPs (30,317 SNP) are in the intergenic region and the intron region.

The present disclosure also provides a molecular marker segment including the molecular marker combination described above. Each of the molecular marker segments includes 40,000 marker segments, and each of the marker segments includes a nucleotide sequence of 100 bp upstream and a nucleotide sequence of 100 bp downstream an SNP, where the SNP is one of the 40,000 SNPs included in the molecular marker combination described in the above technical solution. In the present disclosure, the molecular marker segment preferably includes 74,687 mSNPs, and the numbers of the 74687 mSNPs distributed on chromosomes 1-27 are as follows: 4,434, 4,468, 3,888, 3,672, 3,828, 3,927, 4,168, 4,225, 4,179, 4,063, 3,990, 3,813, 3,794, 3,451, 3,301, 2,933, 2,612, 2,118, 1,743, 1,435, 1,315, 1,076, 794, 684, 429, 315, and 32. In the present disclosure, the 40,000 marker segments and the included 74,687 mSNPs provide an increased number of SNPs, which ensures that the requirements for scientific research applications are met while the application cost is greatly reduced.

The present disclosure also provides a 40K genome-wide liquid chip for Picea abies L. Karst, where the 40K genome-wide liquid chip for Picea abies L. Karst includes the molecular marker combination in the technical solution described above. In the actual detection process, the 40K genome-wide liquid chip for Picea abies L. Karst of the present disclosure can cover 40K marker segments. Each of the segments includes 40,000 marker segments, and each marker segment includes a nucleotide sequence of 100 bp upstream the SNP and a nucleotide sequence of 100 bp downstream the SNP. The 40,000 marker segments correspond one by one to the 40,000 SNPs described in the above technical solution. The 40k marker segment preferably includes 74,687 mSNPs, and the information about the 74,687 mSNPs has been described in the above technical solutions and will not be repeated herein.

The 40K genome-wide liquid chip for Picea abies L. Karst was developed based on GenoBaits® technology within the genotyping by targeted sequencing (GBTS) technology system from Shijiazhuang MolBreeding Biotechnology Co., Ltd. and the 40K genome-wide liquid chip for Picea abies L. Karst was prepared through commissioning Shijiazhuang MolBreeding Biotechnology Co., Ltd.

The 40K genome-wide liquid chip for Picea abies L. Karst evenly covers the genome. This genomic liquid chip has the advantages of rich functional SNPs, low cost, and simple bioinformatical analysis. It can be used in genotyping and conservation of germplasm resources for a variety of spruce including Picea abies L. Karst, as well as in molecular breeding studies such as genetic evaluation of germplasm resources, genetic structure analysis of population, identification of kinship, genetic mapping of quantitative traits (QTL), genome-wide association study (GWAS), and genomic selection (GS), which helps to advance the molecular marker-assisted breeding and genomic selection breeding, shortens the forest tree breeding cycle, improves the selection accuracy and breeding efficiency.

Based on the above advantages, the use of the molecular marker combination, the marker segment, or the 40K genome-wide liquid chip for Picea abies L. Karst described in the above technical solution in the genotyping of spruce, in the conservation of germplasm resources, and in the genetic breeding of Spruce fall within the protection scope of the present disclosure. In the present disclosure, the spruce preferably includes, but is not limited to, Picea abies L. Karst.

In the present disclosure, the spruce genetic breeding is preferably one or more selected from the group consisting of genetic evaluation of germplasm resources, genetic structure analysis of population, identification of kinship, genetic mapping of quantitative traits, genome-wide association study, and genome-wide selection study. More preferably, the genetic breeding of spruce includes genetic evaluation of germplasm resources, genetic structure analysis of populations, identification of kinship, genetic mapping of quantitative traits, genome-wide association study, and genome-wide selection study.

In order to further illustrate the present disclosure, the technical solutions provided by the present disclosure will be described in detail in combination with the accompanying drawings and the examples, which should not be construed as a limitation to the scope of protection.

### Example 1

Provided is a 40K genome-wide liquid chip for Picea abies L. Karst, and the SNP information about the liquid chip is shown as follows:
Table 1 Information about 40,000 SNPs

There were 40,000 SNPs on the 40K genome-wide liquid chip for Picea abies L. Karst, and the information about the 40,000 SNPs is shown in Table 1. Based on the 40,000 SNPs in Table 1, the 40K genome-wide liquid chip for Picea abies L. Karst was developed by using the GenoBaits® technology within the GBTS technology system from Shijiazhuang MolBreeding Biotechnology Co., Ltd., and Shijiazhuang MolBreeding Biotechnology Co., Ltd. was commissioned to prepare the chip.

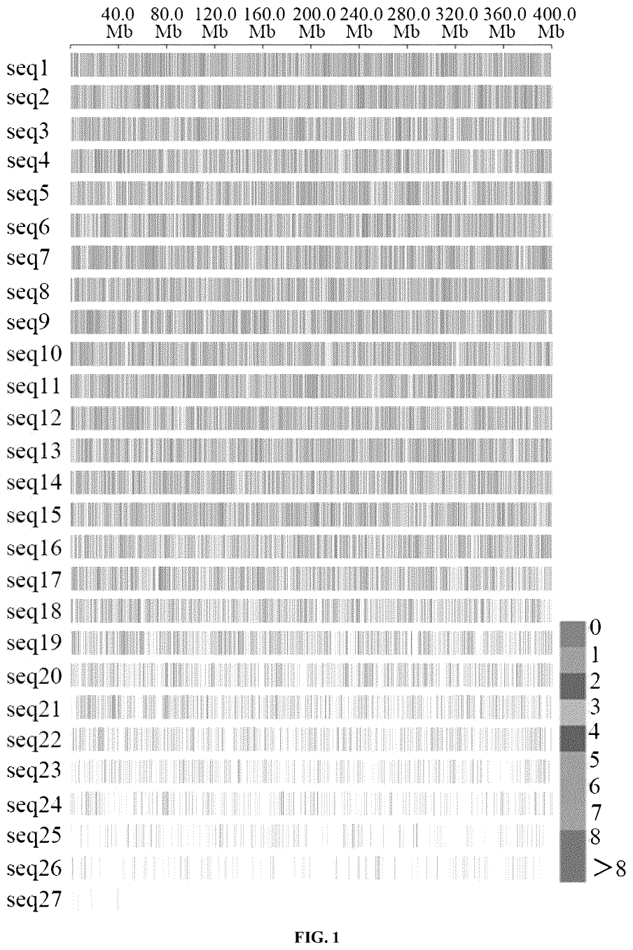

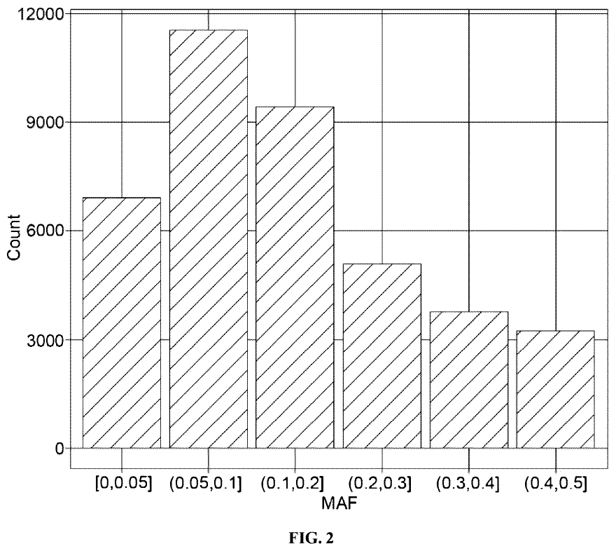

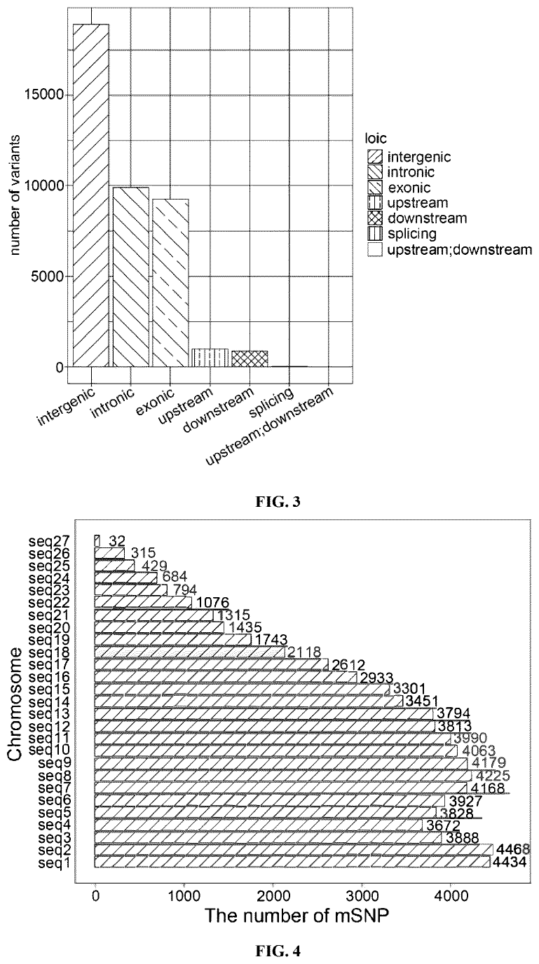

In the development process, the original 24,192 SNPs on the 50K solid-phase chip for Picea abies L. Karst were retained and 26,076 SNPs were optimized to constitute the 40K genome-wide liquid chip for Picea abies L. Karst in the present disclosure, and SNPs after optimization accounted for 51.87% of the original SNPs. In addition, the chip had newly included 218 QTL SNPs significantly associated with the traits covering, for example, wood property of spruce, pest resistance, tree height, and diameter at breast height. Finally, a total of 40,000 high-quality SNPs that had been proven over and over again were obtained and formed a 40K genome-wide liquid chip for Picea abies L. Karst (GenoBaits® Picea abies 40K GBTS panel). In this product, the core SNPs were uniformly distributed across the genome, as shown in FIG. 1. The frequency of minor allele ranged from 0.0-0.5, mainly distributed between 0.05-0.2 (FIG. 2), and 72.44% of the SNPs (30,317 SNPs) were in the intergenic region and the intron region (FIG. 3).

From the above analysis, it can be concluded that the liquid chip in this example had rich information about diversity and was highly representative.

### Example 2

The Picea abies L. Karst has an enormous genome and is expensive for genotype testing. The development of the 40K genome-wide liquid chip for Picea abies L. Karst in Example 1 has established a genotyping system with uniform genome coverage, abundant functional SNPs, low cost, and simple bioinformatical analysis, which has the following information:

The 40K genome-wide liquid chip for Picea abies L. Karst in Example 1 had 40,000 marker segments (each marker segment consisted of a region of 100 bp upstream the SNP and a region of 100 bp downstream the SNP), which could actually provide information about up to 74,688 mSNPs, as shown in FIG. 4. When compared with solid-phase chip, the liquid chip increased the number of SNPs, ensured the satisfaction of the requirements of scientific research and application, and greatly reduced the cost of use.

In addition, the 40K genome-wide liquid chip for Picea abies L. Karst in Example 1 had 218 functional markers (including a plurality of trait co-localization SNPs) covering about 45 common traits of the Picea abies L. Karst, including 30 insect resistance markers, 114 markers associated with wood property and wood anatomical traits, 82 markers associated with germination stage and frost resistance, and 8 markers associated with tree height and diameter at breast height.

Presently, probes have been successfully designed for 170 background SNPs, and the information about the 170 background SNPs is shown in Table 2, 120 of which have biological functions and the rest are transcription factors. Among them, 44 SNPs were associated with germination, 44 SNPs were markers associated with wood properties and anatomical traits, 10 SNPs were associated with growth traits such as the tree height and the diameter at breast height, and 22 were insect resistance markers.

TABLE 2
Information about 170 background SNPs

| Site | Function | Site | Function |
|---|---|---|---|
| MA_10_25927 | Spruce cone rust | MA_20378_51314 | Transcription factor |
| MA_10100176_681 | Budburst stage | MA_20554_10808 | Small ribonucleoproteins |
| MA_10117117_4107 | Budburst stage | MA_208236_3389 | PC5 (principal component 5) |
| MA_10117117_4123 | Budburst stage | MA_214776_1624 | Annual ring breadth in the transition |
| | | | zone |
| MA_101803_21804 | Transcription factor | MA_218924_5577 | Wood density |
| MA_101803_7657 | Transcription factor | MA_2193_67395 | Transcription factor |
| MA_10235390_3386 | Wood density in the | MA_2193_67918 | Transcription factor |
| | transition zone | | |
| MA_10248435_1675 | Budburst stage | MA_2193_67983 | Transcription factor |
| MA_10267291_758 | Transcription factor | MA_222324_1075 | Tree height |
| MA_10267291_759 | Transcription factor | MA_224722_3307 | Budburst stage |
| MA_102821_6808 | Budburst stage | MA_24477_24501 | PC3 (principal component 3) |
| MA_104065_12044 | Transcription factor | MA_25569_28091 | Induced defense response |
| MA_10426882_15768 | Budburst stage | MA_28980_31964 | Transcription factor |
| MA_10426894_3576 | Budburst stage | MA_29081_5066 | Budburst stage |
| MA_10427187_18848 | Budburst stage | MA_29357_4328 | Budburst stage |
| MA_10427214_13968 | Mass index | MA_29357_4423 | Budburst stage |
| MA_10427927_2233 | Budburst stage | MA_31029_9337 | PC3 (principal component 3) |
| MA_10428089_12081 | Transcription factor | MA_33109_11804 | Wood density |
| MA_10428744_29330 | Latewood Annual ring | MA_346723_2241 | Cell number in wood in transition zone |
| | breadth | | |
| MA_10428754_23026 | Diameter at breast | MA_38472_13803 | Annual ring breadth in the transition |
| | height | | zone |
| MA_10428833_21190 | PC3 (principal | MA_3905_29322 | Putative HD2-type histone |
| | component 3) | | deacetylase(Intron) |
| MA_10430403_6537 | Budburst stage | MA_3905_29323 | Putative HD2-type histone |
| | | | deacetylase(Intron) |
| MA_10430455_20871 | Apolipoprotein (Intron) | MA_39589_2732 | Transcription factor |
| MA_10432234_28084 | Tree height | MA_402880_2045 | Cell number in earlywood |
| MA_10432519_8378 | Lirula macrospora | MA_40506_4499 | Tree height |
| MA_10432646_63090 | Wood density | MA_405774_1393 | Budburst stage |
| MA_10432719_1312 | Budburst stage | MA_45312_8906 | Wood density |
| MA_10433411_3386 | Wood density in the | MA_458872_2913 | Budburst stage |
| | transition zone | | |
| MA_10433766_489 | Budburst stage | MA_462319_4322 | Proportion of wood in the transition |
| | | | zone |
| MA_10433886_12255 | PC4 (principal | MA_470173_8006 | Wood density |
| | component 4) | | |
| MA_10434007_77578 | Proportion of latewood | MA_470416_4116 | Transcription factor |
| MA_10434464_1786 | Transcription factor | MA_479900_6145 | Uncharacterized F-box containing |
| | | | protein(Exon) |
| MA_10434579_3654 | Budburst stage | MA_53072_3732 | Growth of Heterobasidiomycetes |
| | | | parvum on sapwood |
| MA_10434624_20686 | Latewood annual ring | MA_533908_7681 | Transcription factor |
| | breadth | | |
| MA_10434805_21408 | Wood density of | MA_53835_9763 | Growth of Heterobasidiomycetes |
| | latewood | | parvum on sapwood |
| MA_10435002_4986 | Mass index | MA_5530_8656 | Budburst stage |
| MA_10435406_13733 | Wood density | MA_5592_18004 | Wood stiffness |
| MA_10435530_8878 | Transcription factor | MA_5592_18009 | Wood stiffness |
| MA_10435530_9090 | Transcription factor | MA_56128_7752 | Growth of Heterobasidiomycetes |
| | | | parvum on sapwood |
| MA_10435542_13930 | Budburst stage | MA_5978_21011 | Growth of Heterobasidiomycetes |
| | | | parvum on sapwood |
| MA_10435574_26634 | Similar to A. thaliana | MA_605776_2978 | Transcription factor |
| | SIGNAL PEPTIDE | | |
| | PEPTIDASE-LIKE | | |
| | 1 (Intron) | | |
| MA_10435921_15593 | Microfibril angle | MA_6240_28733 | Budburst stage |
| MA_10435921_15594 | Microfibril angle | MA_62987_13474 | Wood density of latewood |
| MA_10436058_4902 | Wood density of | MA_63888_14516 | Budburst stage |
| | latewood | | |
| MA_10437131_30678 | Transcription factor | MA_64156_8166 | Budburst stage |
| MA_10437232_6258 | Similar to A. thaliana | MA_6809_4188 | Transcription factor |
| | TCS1 (TRICHOME | | |
| | CELL SHAPE | | |
| | 1)(Exon) | | |
| MA_10437232_6283 | Similar to A. thaliana | MA_6809_4787 | Transcription factor |
| | TCS1 (TRICHOME | | |
| | CELL SHAPE | | |
| | 1)(Exon) | | |
| MA_1045136_4310 | Ratio of earlywood | MA_6809_4852 | Transcription factor |
| | proportion to latewood | | |
| | proportion | | |
| MA_105331_1788 | Transcription factor | MA_692_34946 | Diameter at breast height |
| MA_105837_4049 | Budburst stage | MA_71728_10038 | Transcription factor |
| MA_106297_6413 | Budburst stage | MA_71728_10351 | Transcription factor |
| MA_106297_6469 | Budburst stage | MA_71728_10427 | Transcription factor |
| MA_108198_14639 | Transcription factor | MA_71728_12142 | Transcription factor |
| MA_108198_14919 | Transcription factor | MA_71728_7953 | Transcription factor |
| MA_108198_15578 | Transcription factor | MA_71728_8247 | Transcription factor |
| MA_108198_17077 | Transcription factor | MA_71728_8490 | Transcription factor |
| MA_109804_10278 | Mass index | MA_71728_9678 | Transcription factor |
| MA_115579_9896 | Budburst stage | MA_73095_10356 | Transcription factor |
| MA_118446_4316 | Wood density of | MA_73153_13476 | Transcription factor |
| | earlywood | | |
| MA_120687_5569 | Wood stiffness | MA_73153_13692 | Transcription factor |
| MA_124244_22882 | Transcription factor | MA_73153_13716 | Transcription factor |
| MA_124244_23479 | Transcription factor | MA_733_2747 | Budburst stage |
| MA_12842_2389 | Budburst stage | MA_7494_1582 | Budburst stage |
| MA_12842_2476 | Budburst stage | MA_77821_10373 | Budburst stage |
| MA_12842_2490 | Budburst stage | MA_798143_7416 | Budburst stage |
| MA_12842_2621 | Budburst stage | MA_8052_42129 | Transcription factor |
| MA_1378_4718 | Mass index | MA_80954_29644 | Ratio of earlywood proportion to |
| | | | latewood proportion |
| MA_137887_8617 | Transcription factor | MA_817099_1105 | Annual ring breadth in the transition |
| | | | zone of the wood |
| MA_137887_8941 | Transcription factor | MA_84091_11329 | Growth of Heterobasidiomycetes |
| | | | parvum on sapwood |
| MA_141852_10482 | Budburst stage | MA_86404_1461 | Transcription factor |
| MA_14352_27165 | Induced defense | MA_876922_3616 | Budburst stage |
| | response | | |
| MA_158214_6897 | Wood density | MA_876922_3703 | Budburst stage |
| MA_17884_58584 | Growth of | MA_87899_19291 | Wood stiffness |
| | Heterobasidiomycetes | | |
| | parvum on sapwood | | |
| MA_18142_28884 | Transcription factor | MA_8790100_1384 | Ratio of earlywood proportion to |
| | | | latewood proportion |
| MA_18316_3165 | Induced defense | MA_879384_3894 | Annual ring breadth |
| | response | | |
| MA_184576_18345 | Transcription factor | MA_93306_19178 | Wood density |
| MA_18664_16884 | Transcription factor | MA_940838_2433 | ILITHYIA. A. thaliana HEAT repeat |
| | | | protein involved in immunity(EXON) |
| MA_19575_46274 | Transcription factor | MA_9447489_687 | Cell number in wood in transition zone |
| MA_19575_46392 | Transcription factor | MA_96191_7122 | Ratio of earlywood to latewood |
| MA_19639_30264 | Budburst stage | MA_97119_12277 | Induced defense response |
| MA_19843_35554 | Budburst stage | MA_97571_20468 | Lirula macrospora |
| MA_19950_16139 | PC4 (principal | MA_98424_947 | Ratio of earlywood proportion to |
| | component 4) | | latewood proportion |
| MA_200365_1538 | Budburst stage | MA_9857100_676 | Budburst stage |
| MA_20321_44812 | Wood density of | MA_99004_17108 | Mass index |
| | earlywood | | |
| MA_20322_28351 | Annual ring breadth | MA_9964484_611 | Budburst stage |

### Application Example 1

### Materials and Methods

Collection of experimental sample: 239 plants of the Picea abies L. Karst, from the Research Institute of Forestry of Xiaolong Mountain in Tianshui, Gansu Province, were selected, and the leaves of that year were collected. Genomic DNA was extracted using a DNA extraction kit developed by Shijiazhuang MolBreeding Biotechnology Co., Ltd. The 40K genome-wide liquid chip for Picea abies L. Karst in Example 1 was used for sequencing to obtain the genotype data.

The results showed that the 40K genome-wide liquid chip for Picea abies L. Karst gave a detection rate of 90.00%-99.83% in this population (bases ATCG but not NA were detected), and the average detection rate was 97.84%.

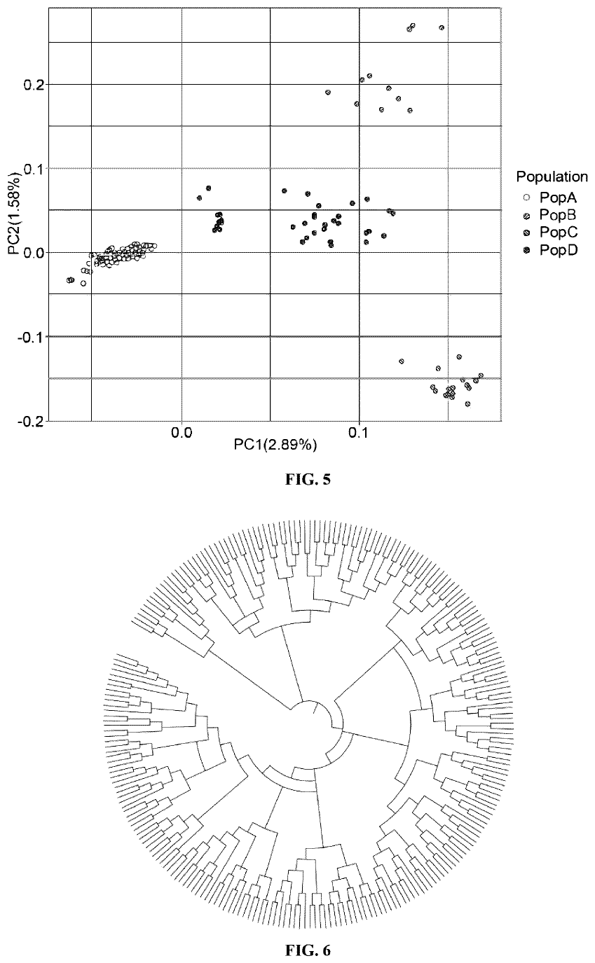

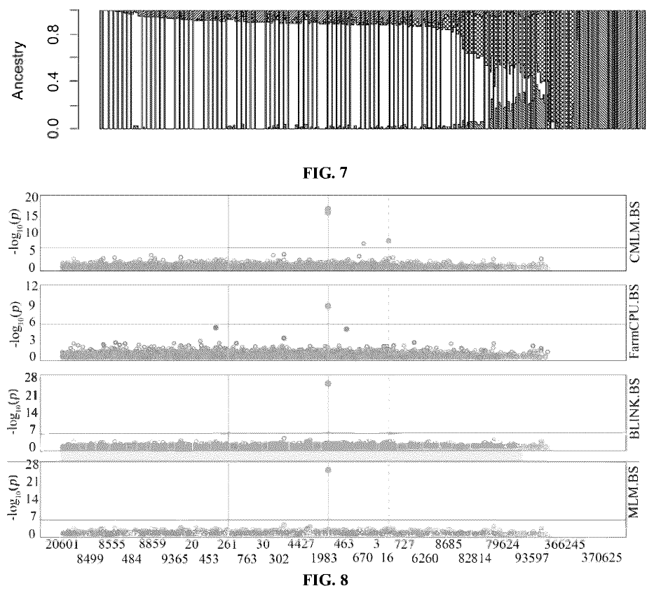

Analysis of population structure: based on the typing data from the 40K liquid chip for the population, the principal component, the phylogenetic tree, and the genetic structure of the population were analyzed (FIG. 5-FIG. 7), and the specific method was as follows: MEGA software was used to construct NJ trees by using a neighbor-joining method; the R language was used for principal component analysis (PCA) of the population, the software Admixture was used to infer the population structure, the subpopulation number (K value) of the samples was assumed as 1-10, and then clustering was conducted. The optimal subpopulation number was determined based on the cross-validation error (Cross-validation Error, CV error) rate, and the K value with the smallest cross-validation error rate corresponded to the optimal subpopulation number.

It can be concluded from the results of FIG. 5-FIG. 7 that the 40K genome-wide liquid chip for Picea abies L. Karst in Example 1 was effective in distinguishing between the Picea abies L. Karst population of indefinite origins (unclear pedigree), the 239 samples were divided into 4 distinct subpopulations, and the subpopulations were highly distinguished.

### Application Example 2

This example discloses the application of the 40K liquid chip for Picea abies L. Karst in the genome-wide association study of the Picea abies L. Karst, including the following steps:

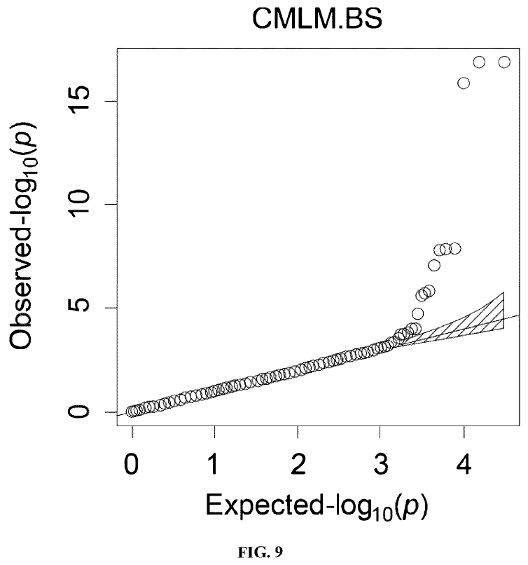

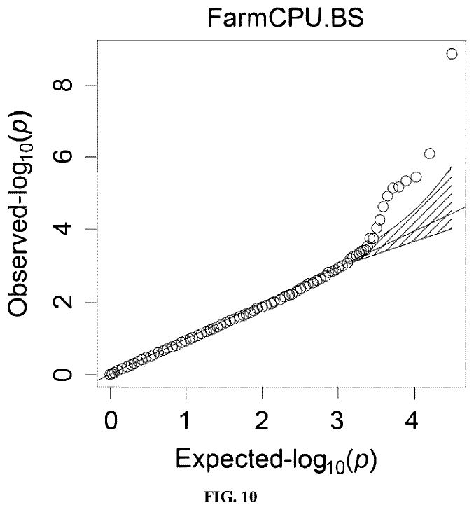

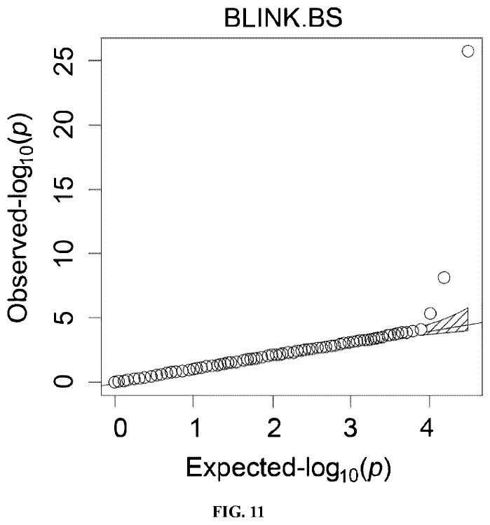

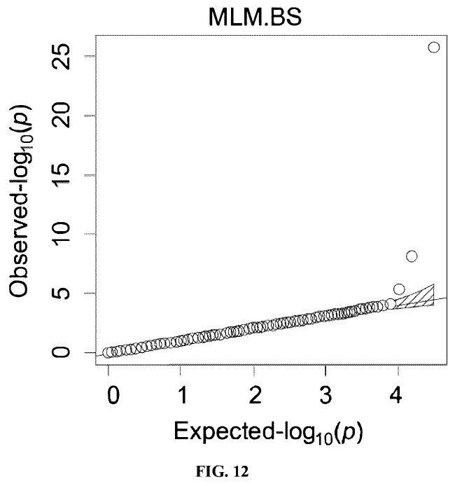

465 Picea abies L. Karst clones were taken as the research object, performing sequencing using the 40K liquid chip for Picea abies L. Karst to obtain the genotype data of the samples, the genotyping results were subjected to quality control. After genotype imputation of missing genotypes of this population using the software Beagler, the SNPs having a minimum allele frequency of less than 0.05 were removed to finally obtain 31,285 SNP markers for 465 individual clones. Subsequently, the screened SNPs, together with the leaf bud germination grade data on the Picea abies L. Karst as measured, were subjected to genome-wide association study analysis using the four models of CMLM, FarmCPU, BLINK, and MLM 4 in the software GAPIT. The results are shown in FIG. 8-12. Ultimately, it was found that the marker SNPs that were significantly associated with the germination grade trait of Picea abies L. Karst included 10 markers of seq_4184789197, seq2_271222929, seq1_1135558448, and seq4_141520477, seq2_205926727, seq1_496965149, seq4_355356241, seq1_027355281, seq9_386733646, and seq7_97879342.

Although the above examples provide a detailed description of the present disclosure, they are merely part of, but not all of the embodiments of the present disclosure, and other embodiments may also be obtained without creative efforts, all of which fall within the protection scope of the present disclosure.

LENGTHY TABLES
The patent application contains a lengthy table section. A copy of the table is available in electronic form from the USPTO web site (https://seqdata.uspto.gov/docdetail?docId=US20260117320A1). An electronic copy of the table will also be available from the USPTO upon request and payment of the fee set forth in 37 CFR 1.19(b)(3).

What is claimed is:

1. A molecular marker combination for genotyping of Picea abies L. Karst, wherein the molecular marker combination comprises 40,000 single nucleotide polymorphisms (SNPs), and
the 40,000 SNPs have information shown in the table below:

2. A molecular marker segment comprising the molecular marker combination of claim 1, wherein the molecular marker segments comprises 40,000 marker segments, each of the marker segment comprises a 100 bp nucleotide sequence upstream of the SNP and a 100 bp nucleotide sequence downstream of the SNP, wherein the SNP is one of the 40,000 SNPs comprised in the molecular marker combination of claim 1.
Lengthy table referenced here
US18814960-20260430-T00002
Please refer to the end of the specification for access instructions.

3. The molecular marker segment according to claim 2, wherein the molecular marker segment comprises 74,687 multiple single nucleotide polymorphisms (mSNPs).

4. A 40K genome-wide liquid chip for Picea abies L. Karst, wherein the 40K genome-wide liquid chip for Picea abies L. Karst comprises the molecular marker combination of claim 1.

5. A method for genotyping spruce, comprising a step of applying the 40K genome-wide liquid chip for Picea abies L. Karst of claim 4.

6. A method for conserving germplasm resources, comprising a step of applying the 40K genome-wide liquid chip for Picea abies L. Karst of claim 4.

7. A method for genetically breeding spruce, comprising a step of applying the 40K genome-wide liquid chip for Picea abies L. Karst of claim 4.

8. The method according to claim 7, wherein the genetic breeding of spruce comprises one or more selected from the group consisting of genetic evaluation of germplasm resources, genetic structure analysis of a population, identification of kinship, genetic mapping of quantitative traits, genome-wide association study, and genome-wide selection study.
